(19) United States
(12) Patent Application Publication
(10) Pub. No.: US 2025/0123260 A1
(43) Pub. Date: Apr. 17, 2025

(54) IN PLANE TISSUE STRETCHING SYSTEM AND METHOD

(71) Applicant: Purdue Research Foundation, West Lafayette, IN (US)

(72) Inventors: Luis Solorio, West Lafayette, IN (US); Hyowon Lee, West Lafayette, IN (US); Angel Guillermo Enriquez, West Lafayette, IN (US); Madison Mckensi Howard, West Lafayette, IN (US); Chun-Wei Hsu, San Diego, CA (US); Sarah Libring, Franklin, TN (US)

(21) Appl. No.: 18/913,378

(22) Filed: Oct. 11, 2024

Related U.S. Application Data
(60) Provisional application No. 63/544,021, filed on Oct. 13, 2023.

(51) Int. Cl. G01N 33/483; G01N 3/04; G01N 3/06; G01N 3/08

(52) U.S. Cl. CPC G01N 33/4833; G01N 3/04; G01N 3/066; G01N 3/08; G01N 2203/0003; G01N 2203/0017; G01N 2203/0075; G01N 2203/0252; G01N 2203/0282; G01N 2203/0286; G01N 2203/0676

(57) ABSTRACT

A tissue stretching system includes a motor, a first pillar, a second pillar, a force sensor, and an imaging device. The tissue stretching system may be provided as a 2D axial tissue stretcher that may monitor changes in mechanical properties over time. Tissues may be coated onto the first pillar and/or the second pillar. In a specific example, the first pillar and/or the second pillar may be a PDMS (Polydimethylsiloxane) structure configured to probe the mechanical properties in between a designed gap of the first pillar and the second pillar. The motor of the tissue stretching system includes a microcontroller. The motor of the tissue stretching system may have high resolution capabilities to provide the appropriate strain to the sample. The tissue stretching system then determines the actual amount of strain applied to the sample.

### CROSS REFERENCE TO RELATED APPLICATIONS

This application claims the priority benefit of U.S. Provisional Patent application No. 63/544,021, filed Oct. 13, 2023, the contents of which is incorporated herein by reference in its entirety.

### FIELD

The disclosure generally relates to microscopy systems and, more particularly, to systems configured to capture images of cell progression.

### INTRODUCTION

This section provides background information related to the present disclosure which is not necessarily prior art.

Simultaneous mechanical stimulation and sensing while imaging biological cells remains a challenging endeavor for the field of tissue engineering. More specifically, determining the mechanical properties of tissue is a known challenge in this industry. The ability to detect any changes in the mechanical properties of tissues caused by the seeded cells is vital information for understanding cell response to dynamic environments encountered in the heart, lungs, bones, etc. By simultaneously imaging the morphological effects mechanical stimulation has on these cells, researchers can capture the progression of these changes. However, known mechanical stimulation platforms rely on synthetic, non-physiologically relevant materials to seed cells on. Additionally, many of these known systems do not have the capability to sense force in real-time while being imaged using a microscope.

Accordingly, there is a continuing need for a tissue-stretching system having a stretching platform with high-resolution force sensing capabilities and tailored for microscopy cell culture experiments.

### SUMMARY

In concordance with the present disclosure, a tissue-stretching system having a stretching platform with high-resolution force sensing capabilities and tailored for microscopy cell culture experiments, has surprisingly been discovered.

A tissue stretching system includes a stretching frame, a motor, a first pillar, a second pillar, a force sensor, and an imaging device. The tissue stretching system may be provided as a 2D axial tissue stretcher that may monitor changes in mechanical properties over time. In certain circumstances, tissues may be coated onto the first pillar and/or the second pillar. In a specific example, the first pillar and/or the second pillar may be a PDMS (Polydimethylsiloxane) structure configured to probe the mechanical properties in between a designed gap of the first pillar and the second pillar. The motor of the tissue stretching system may include a microcontroller. The motor of the tissue stretching system may have high resolution capabilities to provide the appropriate strain to the sample.

The tissue stretching system may be used in various ways. For instance, the tissue stretching system may be used according to a method. The method may include a step of coating a first pillar and/or a second pillar with a biological substrate, such as an extracellular matrix (ECM) protein or tissue sample. These ECM proteins may be fibronectin, collagen, or fibrin. Next, the biological substrate coated first pillar and/or second pillar may be fluorescently tagged. Afterwards, the fibronectin-coated first pillar and/or second pillar may be coupled to the stretching frame. A baseline measurement of strain may be determined between the first pillar and the second pillar. Then, a strain may be applied by the motor moving the first pillar from the second pillar. Next, the amount of displacement from the motor may be determined to obtain the amount of strain applied to a sample. A skilled artisan may select other suitable ways for using the tissue stretching system, within the scope of the present disclosure. It is contemplated that the order of the steps of the method may be rearranged.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a top perspective view of a tissue stretching system, according to one embodiment of the present disclosure;

FIG. 2 is a top plan view of the tissue stretching system, as shown in Claim 1, according to one embodiment of the present disclosure;

FIG. 3 is an enlarged top perspective view of the first pillar, the second pillar, and the sample, as shown call-out A in FIG. 2, according to one embodiment of the present disclosure;

FIG. 4 is a microscopic image of a fibronectin coating on the first pillar and the second pillar, taken from call-out B in FIG. 3, according to one embodiment of the present disclosure;

FIG. 5 is a bottom perspective view of the tissue stretching system, as shown in FIGS. 1 and 2, according to one embodiment of the present disclosure;

FIG. 6 is a front elevational view of a tissue stretching system, further depicting an imaging device disposed substantially adjacent to the first pillar and the second pillar, according to one embodiment of the present disclosure;

FIG. 7 is a microscopic image of an unstrained sample, according to one embodiment of the present disclosure;

FIG. 8 is a microscopic image of a ˜40% strained sample, according to one embodiment of the present disclosure;

FIG. 9 is a line graph of the strain rate equaling 0.006 s⁻¹ where 20% strain was applied at 0.3 Hz, according to one embodiment of the present disclosure; and

FIG. 10 is a flow chart of a method for using the tissue stretching system, according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the FIG. is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

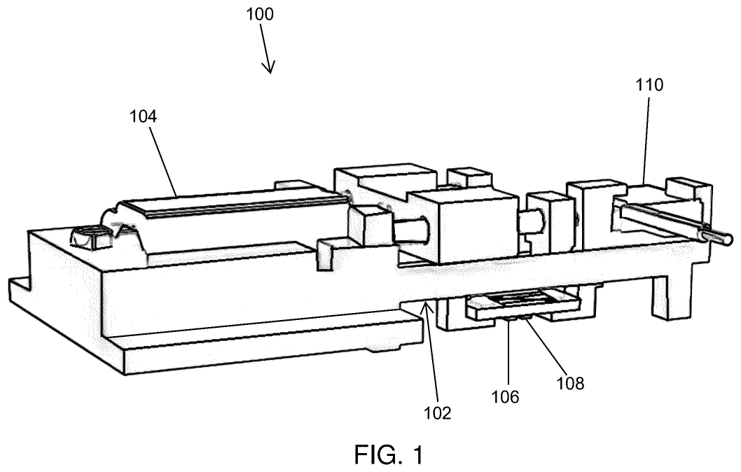

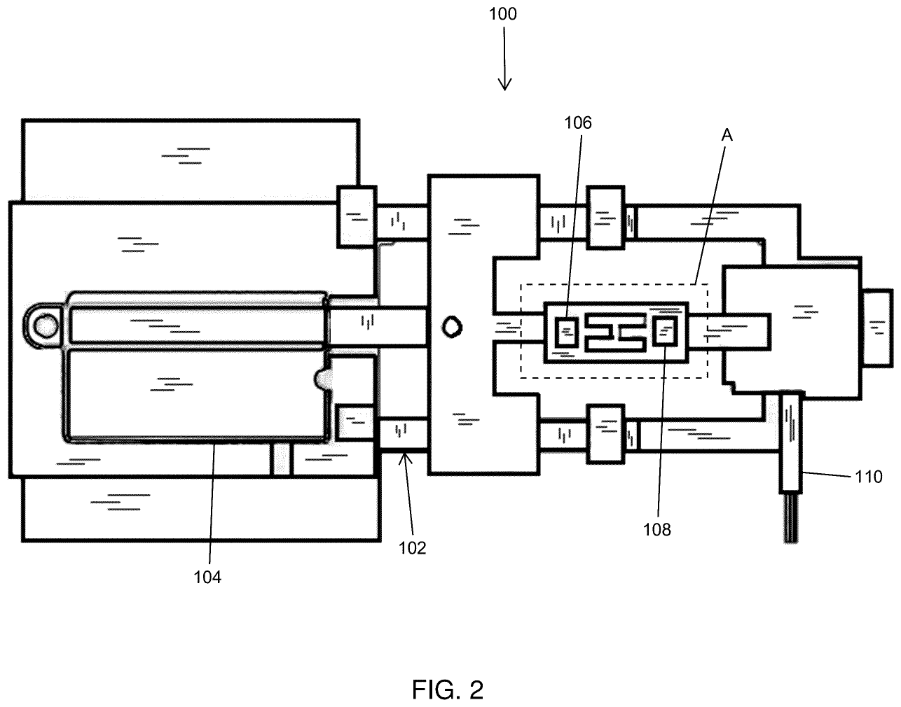

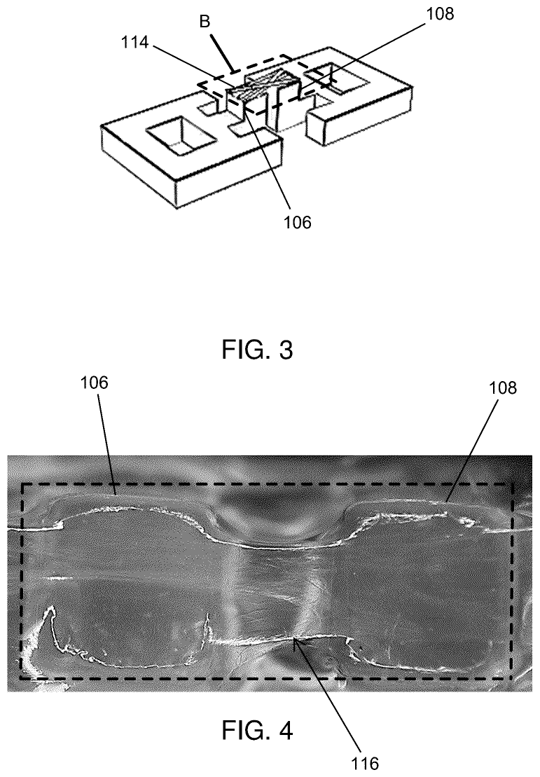

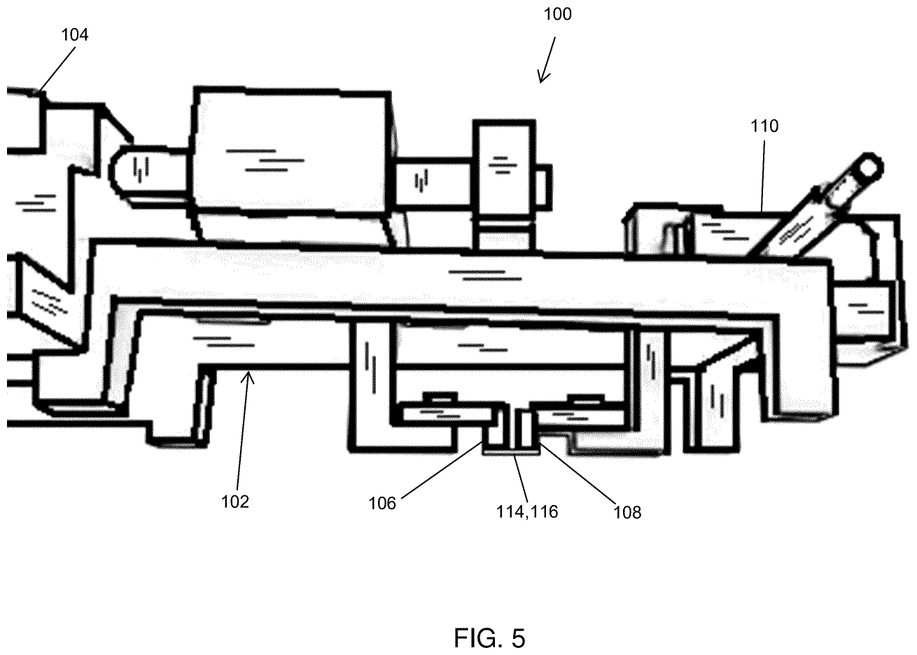

As shown in FIGS. 1-2, and 5, the tissue stretching system 100 includes a stretching frame 102, a motor 104, a first pillar 106, a second pillar 108, a force sensor 110, and an imaging device 112. The tissue stretching system 100 may be provided as a 2D axial tissue stretcher that may monitor changes in mechanical properties over time. In certain circumstances, as shown in FIG. 3, a tissue sample 114 may be coated onto the first pillar 106 and/or the second pillar 108. It is contemplated that more than two pillars may be utilized in the tissue stretching system 100. In a specific example, the first pillar 106 and/or the second pillar 108 may be a PDMS (Polydimethylsiloxane) structure 106,108 configured to probe the mechanical properties in between a designed gap of the first pillar 106 and the second pillar 108. In a specific example, the PDMS structure 106,108 may be at least partially coated with a biological substrate 116, such as an extracellular matrix (ECM) protein or tissue sample. These ECM proteins may be fibronectin, collagen, or fibrin. The biological substrate 116 may be disposed across the gap to provide a base layer for the sample to be coupled to. It is also contemplated for other materials to be disposed as a base layer across the gap, such as hydrogels, tendons, and/or ligaments. A skilled artisan may select other suitable materials to provide a base layer for the sample, within the scope of the present disclosure. The motor 104 of the tissue stretching system 100 may include an arduino-controlled motor with high resolution to provide the appropriate strain to the PDMS structure 106,108. In another specific example, the motor 104 may include a first coupling mechanism, such as a first hook attachment at the end of an actuator that may couple the motor 104 to the PDMS structure 106,108. One skilled in the art may select other suitable ways to couple the motor 104 to the PDMS structure 106,108. The force sensor 110 may be disposed substantially opposite the motor 104 from the PDMS structure 106,108. In other words, the PDMS structure 106,108 may be disposed between the motor 104 and the force sensor 110. The force sensor 110 may include a micro-force sensor capable of detecting any mechanical property changes of the matrix over time. In a specific example, the force sensor 110 may include a second coupling mechanism, such as a second hook attachment that may couple the force sensor 110 to the PDMS structure 106,108. In a more specific example, the first coupling mechanism and the second coupling mechanism may be disposed on opposing side of the PDMS structure 106,108. One advantage of a hook coupling mechanism is to militate against having the electronics, the force sensor 110, and the motor 104 in contact with the PBS structure in the petri dish, as well as decreasing the distance from a microscope focal plane to the region of interest. In a specific example, the force sensor 110 may have a resolution between around 1-50 μN. In another specific example, the force sensor 110 may have a maximum force between around 0.5-5N. In a more specific example, the force sensor 110 may have around a 19 μN resolution and a maximum force of 1 N. The high resolution of the force sensor 110 enables measuring the changes in normal force (FN) over time.

One contemplated technique of strain verification is to calculate the global strain of the tissues by measuring the displacement of the axial motor 104. Another technique is to first enable optical measurement of engineering strain at a region of interest. The optical measurement may require fluorescent tagging of the fibronectin coated PDMS structure 106,108. For instance, provided as a non-limiting example, the fluorescent tagging may include utilizing alexa fluor 488-conjugated wheat germ agglutinin protein (WGA) diluted 1:50 in 1× PBS immersing the structure for 30 minutes. With continued reference to the non-limiting example, fluorescence was confirmed by imaging using an upright Zeiss LSM 800 confocal microscope (Carl Zeiss Microscopy) and a 10× Plan-Neofluar (NA=0.3) objective. Fiducial lines were photobleached onto fibronectin using the crop feature in the ZenBlue software (Carl Zeiss Microscopy). Regions of interest (ROI) consisted of 20 μm width and the entire length of the image window. They were photobleached by setting the laser at 100% power and performing a z-stack scan across the thickness of the sample at 10 μm increments. Each ROI was separated by 50 μm until creating 5 fiducial lines in the sample. One skilled in the art may select other suitable ways for enabling the optical measurement of the PDMS structure 106,108, within the scope of the present disclosure.

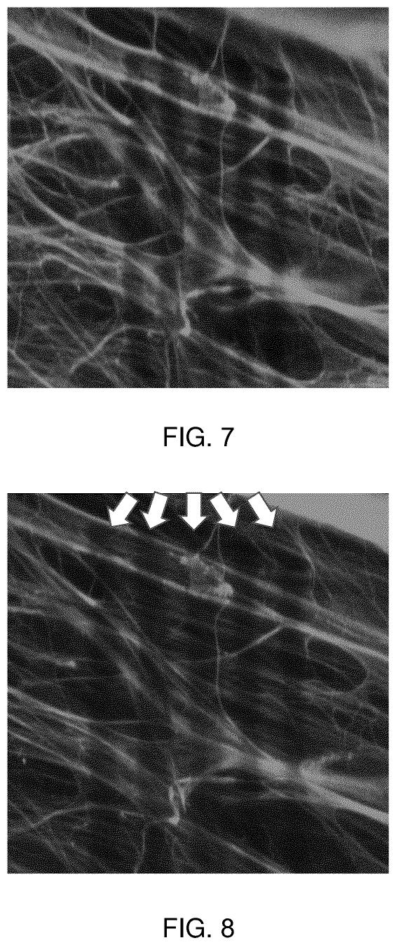

Once the fibronectin coated PDMS structure 106,108 is coupled to the stretching frame 102, the strain applied to the fibronectin strands may be verified by measuring the change in distance of the fiducial markers. As shown in FIG. 7, a baseline measurement of the fiducial markers may first be obtained. Then, as shown in FIG. 8, a displacement may be applied to determine the engineering strain in the region of interest of the sample. With this, the amount of displacement needed by the motor 104 to apply an engineering strain at the sample of interest may be identified. For instance, provided as a non-limiting example, a 1.2 mm displacement of the PDMS structure 106,108 by the motor 104 was shown to result in 40% strain on the sample.

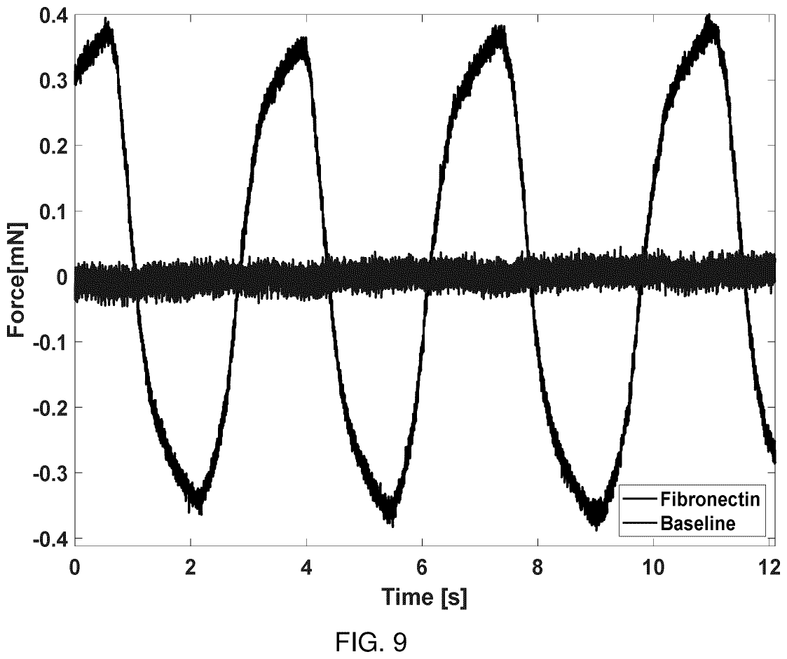

As shown in FIG. 9, the force sensor 110 in the tissue stretching system 100 may have the ability to capture the progression of the tissue's mechanical properties over time. Without being bound to any particular theory, it is believed that culturing cells on these tissues, while being cyclically stretched, may elicit a response to modify their extracellular environment. In a specific example, the present disclosure may be suitable to demonstrate how breast cancer cells exhibited aspects of dormance when exposed to a dynamic environment when compared to their static counterparts. A skilled artisan may select other suitable applications for this system, within the scope of the present disclosure.

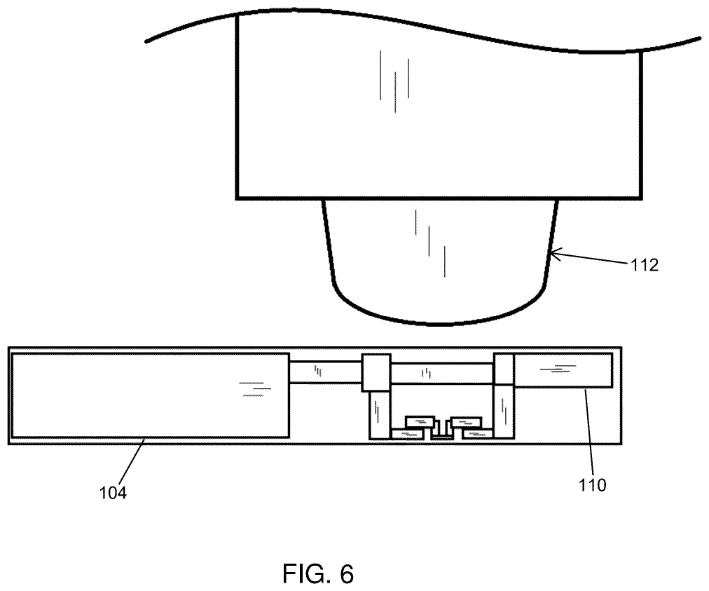

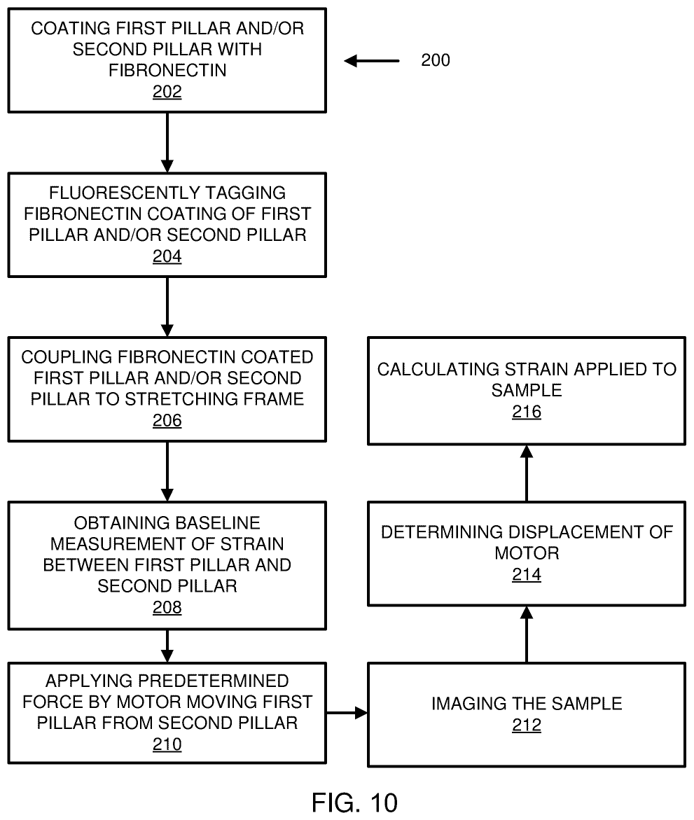

The tissue stretching system 100 may be used in various ways. For instance, as shown in FIG. 10, the tissue stretching system 100 may be used according to a method 200. The method 200 may include a step 202 of coating a first pillar 106 and/or a second pillar 108 with a biological substrate 116. Next, the biological substrate-coated first pillar 106 and/or second pillar 108 may be fluorescently tagged. Afterwards, the biological substrate-coated first pillar 106 and/or second pillar 108 may be coupled to the stretching frame 102. A baseline measurement of strain may be determined between the first pillar 106 and the second pillar 108. Then, a strain may be applied by the motor 104 moving the first pillar 106 from the second pillar 108. While the strain is being applied, the imaging device 112 may take an image of the sample, as shown in FIG. 6. In a specific example, the imaging device 112 may include a microscope, such as a fluorescence microscope. Next, the amount of displacement from the motor 104 may be determined to obtain the actual amount of strain applied to a sample. The force sensor 110 may record the force from the sample during stretching and imaging to aid in determining mechanical properties of the biological substrate 116. A skilled artisan may select other suitable ways for using the tissue stretching system 100, within the scope of the present disclosure. It is contemplated that the order of the steps of the method may be rearranged.

Advantageously, the tissue stretching system 100 may enable mechanical characterization of the tissues while being stretched uniaxially as well as being designed for microscopy cell culture. Desirably, information obtained from this multimodal system of the present disclosure may help understand the progression of cancer cells when they metastasize from static microenvironments to dynamic environments.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions, and methods can be made within the scope of the present technology, with substantially similar results.

What is claimed is:

1. A tissue stretching system configured to actuate and measure an applied strain and force to a sample, the system comprising:
a first pillar coupled to a motor;
a second pillar coupled to a force sensor, wherein there is a gap between the first pillar and the second pillar; and
an imaging device disposed substantially adjacent to at least one of the first pillar and the second pillar.

2. The tissue stretching system of claim 1, wherein at least one of the first pillar and the second pillar include Polydimethylsiloxane (PDMS).

3. The tissue stretching system of claim 2, wherein at least one of the first pillar and the second pillar are at least partially coated with a biological sample.

4. The tissue stretching system of claim 3, wherein the biological sample includes at least one of fibronectin, collagen, and fibrin.

5. The tissue stretching system of claim 3, wherein the biological sample is disposed across the gap.

6. The tissue stretching system of claim 5, wherein the biological sample is one of a hydrogel, a tendon, and a ligament.

7. The tissue stretching system of claim 2, wherein at least one of the first pillar and the second pillar are fluorescently tagged.

8. The tissue stretching system of claim 1, wherein each of the first pillar and the second pillar are disposed between the force sensor and the motor.

9. The tissue stretching system of claim 8, wherein the motor is coupled to the first pillar with a first hook.

10. The tissue stretching system of claim 8, wherein the force sensor is coupled to the second pillar with a second hook.

11. The tissue stretching system of claim 1, wherein the force sensor has a resolution between around one to fifty micronewtons.

12. The tissue stretching system of claim 11, wherein the force sensor has a resolution around nineteen micronewtons.

13. The tissue stretching system of claim 1, wherein the force sensor has a maximum force between around half a newton to around five newtons.

14. The tissue stretching system of claim 13, wherein the force sensor has a maximum force of around one newton.

15. A method of using a tissue stretching system, the method comprising the steps of:
coating at least one of a first pillar and a second pillar with a biological substrate;
coupling the first pillar and the second pillar to the stretching frame;
determining a baseline measurement of strain between the first pillar and the second pillar;
applying a strain by the motor moving the first pillar from the second pillar;
calculating an amount of displacement from the motor;
recording an amount of force from the force sensor; and
determining an amount of strain applied to a sample.

16. The method of claim 15, further comprising a step of imaging the sample during the step of applying the strain by the motor.

17. The method of claim 15, further comprising a step of fluorescently tagging at least one of the first pillar and the second pillar.

18. The method of claim 17, wherein the step of fluorescently tagging at least one of the first pillar and the second pillar includes disposing the at least one of the first pillar and the second pillar in solution with a wheat germ agglutinin (WGA) protein.
